(19) United States
(12) Patent Application Publication
(10) Pub. No.: US 2017/0027555 A1
(43) Pub. Date: Feb. 2, 2017

(54) SURGICAL RETRACTOR

(71) Applicant: EBI, LLC, Parsippany, NJ (US)

(72) Inventors: Benjamin PAUMIER, Morristown, NJ (US); Robert NEAL, Morris Plains, NJ (US)

(21) Appl. No.: 15/303,901

(22) PCT Filed: Apr. 16, 2014
(86) PCT No.: PCT/US2014/034353

§ 371 Date: Oct. 13, 2016

(51) Int. Cl. A61B 17/02

(52) U.S. Cl. CPC A61B 17/0293; A61B 2017/00473

(57) ABSTRACT

A surgical retractor includes a retractor frame, a plurality of blades for engaging tissue, and a coupling arrangement. Each blade of the plurality of blades defines a longitudinal blade axis. The coupling arrangement is carried by the retractor frame and releasably couples a first blade of the plurality of blades with the retractor frame through translation of the first blade in a direction generally perpendicular to the longitudinal blade axis of the first blade.

### FIELD

The present disclosure relates to a surgical retractor, and more particularly to a surgical retractor including front-loading blades.

### BACKGROUND

This section provides background information related to the present disclosure which is not necessarily prior art.

Various devices and associated methods are known for providing access to portions of the human body during a surgical procedure. Such devices include, for example, forceps, dilators and retractors. A surgical retractor may include at least two blades that can be inserted into the human body through an incision during a surgical procedure. The blades can be separated or retracted in order to open or otherwise enlarge the incision, thus providing improved access to the surgical site. While known retractors have proven to be acceptable for their intended purposes, a continuous need for improvement in the art remains.

### SUMMARY

This section provides a general summary of the disclosure, and is not a comprehensive disclosure of its full scope or all of its features.

According to one particular aspect, the present disclosure provides a surgical retractor. The surgical retractor includes a retractor frame, a plurality of blades for engaging tissue, and a coupling arrangement.

Each blade of the plurality of blades defines a longitudinal blade axis. The coupling arrangement is carried by the retractor frame and releasably couples a first blade of the plurality of blades with the retractor frame through translation of the first blade in a direction generally perpendicular to the longitudinal blade axis of the first blade.

According to another particular aspect, the present disclosure provides a surgical retractor. The surgical retractor includes a retractor frame, a plurality of blades for engaging tissue, and a coupling arrangement. Each blade of the plurality of blades defines a longitudinal axis and includes a mounting portion having a pair of blade arms. The coupling arrangement is carried by the retractor frame and releasably couples a first blade of the plurality of blades with the retractor frame through translation of the first blade in a direction generally perpendicular to the longitudinal blade axis of the first blade. The coupling arrangement including a pair of coupling arms moveable relative to one another in a direction generally perpendicular to the longitudinal axis of the first blade.

According to yet another particular aspect, the present disclosure provides a surgical retractor. The surgical retractor includes a retractor frame, a plurality of blades for engaging tissue, and a corresponding plurality of coupling arrangements carried by the retractor frame. Each blade of the plurality of blades defines a longitudinal axis and includes a mounting portion having a pair of blade arms. Each coupling arrangement of the corresponding plurality of coupling arrangement is operative to releasably couple a corresponding blade of the plurality of blades with the retractor frame through translation of the corresponding blade in a direction generally perpendicular to the longitudinal axis of the corresponding blade.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a surgical retractor in accordance with the principles of the present disclosure.

FIG. 2 is a top view of the surgical retractor of FIG. 1.

FIG. 3 is a front view of a coupling arrangement of the surgical retractor of FIG. 1, the coupling arrangement shown in a first configuration.

FIG. 4 is a front view of the coupling arrangement of FIG. 3, the coupling arrangement shown in a second configuration.

FIG. 5 is a cross-sectional view of the connector assembly of FIG. 4, taken along the line 5-5 of FIG. 4.

FIG. 6 is a perspective view of another surgical retractor in accordance with the principles of the present disclosure.

FIG. 7 is a top view of the surgical retractor of FIG. 6.

FIG. 8A is a cross-sectional view of a coupling arrangement of the surgical retractor of FIG. 6 taken along the line 8A-8A of FIG. 6, the coupling arrangement shown in a first configuration.

FIG. 8B is a cross-sectional view of the coupling arrangement of the surgical retractor of FIG. 6 taken along the line 8B-8B of FIG. 6, the coupling arrangement shown in a first configuration.

FIG. 9A is a cross-sectional view similar to FIG. 8A, the coupling arrangement shown in a second configuration.

FIG. 9B is a cross-sectional view similar to FIG. 8B, the coupling arrangement shown in a second configuration.

Corresponding reference numerals indicate corresponding parts throughout the several views of the drawings.

### DETAILED DESCRIPTION

Example embodiments will now be described more fully with reference to the accompanying drawings.

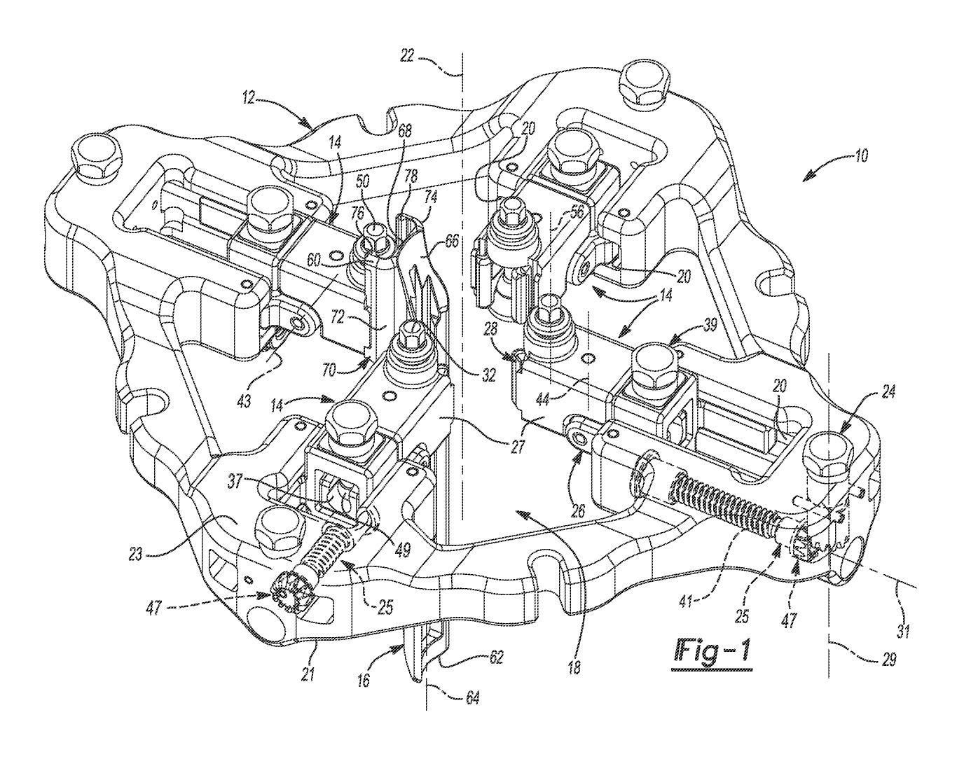

With initial reference to FIG. 1 of the drawings, a surgical retractor constructed in accordance with the principles of the present disclosure is illustrated and identified at reference character 10. According to one exemplary use, the retractor 10 may be used for accessing a lumbar intervertebral disc space (not shown) during a surgical procedure in order to facilitate the placement of an implant into the lumbar intervertebral disc space. It will be appreciated, however, that the retractor 10 may be adapted for use in many different applications, such as accessing the posterior of a vertebra body or disc space, accessing a cervical vertebra body or disc space, or accessing various other portions of the human body during a surgical procedure.

The surgical retractor 10 may include a frame 12, a coupling arrangement 14, and a blade 16. For clarity, the surgical retractor 10 is shown with one blade 16; however, in one configuration the retractor 10 may include four coupling arrangements 14 and four blades 16. It will also be appreciated that the retractor may include more or less than four coupling arrangements 14 and more or less than four blades 16 within the scope of the present disclosure. In this regard, the number of coupling arrangements 14 may be equal to the number of blades 16.

The frame 12 may be generally ring-shaped, defining a central opening 18. The central opening 18 may define a first longitudinal axis 22 and may allow for viewing a surgical site. In this regard, a lower side 21 (relative to the view in FIG. 1) of the frame 12 may generally face the surgical site, while an upper side 23 of the frame 12 may generally face away from the surgical site. In one configuration, the frame 12 may further include at least one pair of opposing grooves or channels 20 for mounting the coupling arrangement 14 to the frame 12. The channels 20 may extend in a direction substantially perpendicular to the first longitudinal axis 22.

The coupling arrangement 14 may be adjustably mounted to the frame 12. The location of the coupling arrangement 14 relative to the frame 12 can be selectively adjusted in a direction substantially perpendicular to the first longitudinal axis 22 by sliding or otherwise moving the coupling arrangement 14 within the channels 20. In this regard, the retractor 10 may include a first driving member 24 and a second driving member 25. The first driving member 24 may include a first gear portion 45 and may be mounted to the frame 12 for rotation about a first axis of rotation 29. The first axis of rotation 29 may be substantially parallel to the first longitudinal axis 22. The second driving member 25 may include a second gear portion 47 and a threaded portion 41. The second driving member 25 may be mounted to the frame 12 and threadably mounted to the coupling arrangement 14 for rotation about a second axis of rotation 31. The second axis of rotation 31 may be substantially perpendicular to the first axis of rotation 29. The first gear portion 45 may be meshingly engaged with the second gear portion 47, such that rotation of the first driving member 24 causes rotation of the second driving member 25, including causing the threaded portion 41 to threadably engage the coupling arrangement 14. Threaded engagement of the threaded portion 41 to the coupling arrangement 14 may cause the coupling arrangement to move within the channels 20 of the frame 12.

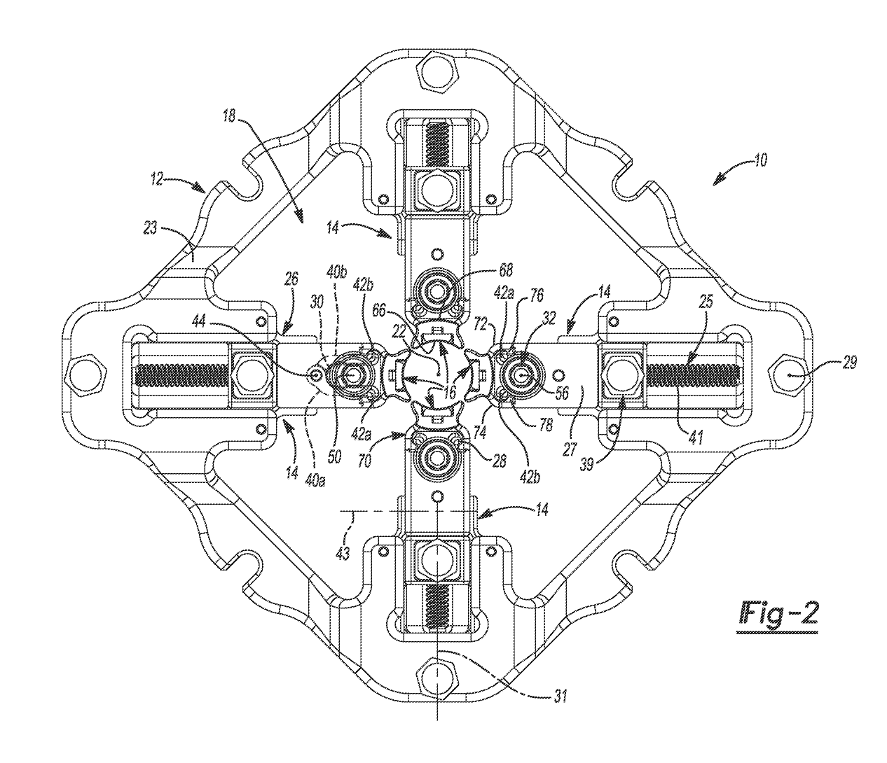

The coupling arrangement 14 may include a carrier 26, a connector housing 27, a connector 28, a biasing member 30, and a locking element 32. In an assembled configuration, the carrier 26 may be adjustably coupled to the frame 12. As illustrated in FIGS. 1 and 2, in one configuration, the carrier 26 may be adjustably disposed within the channels 20, as described above.

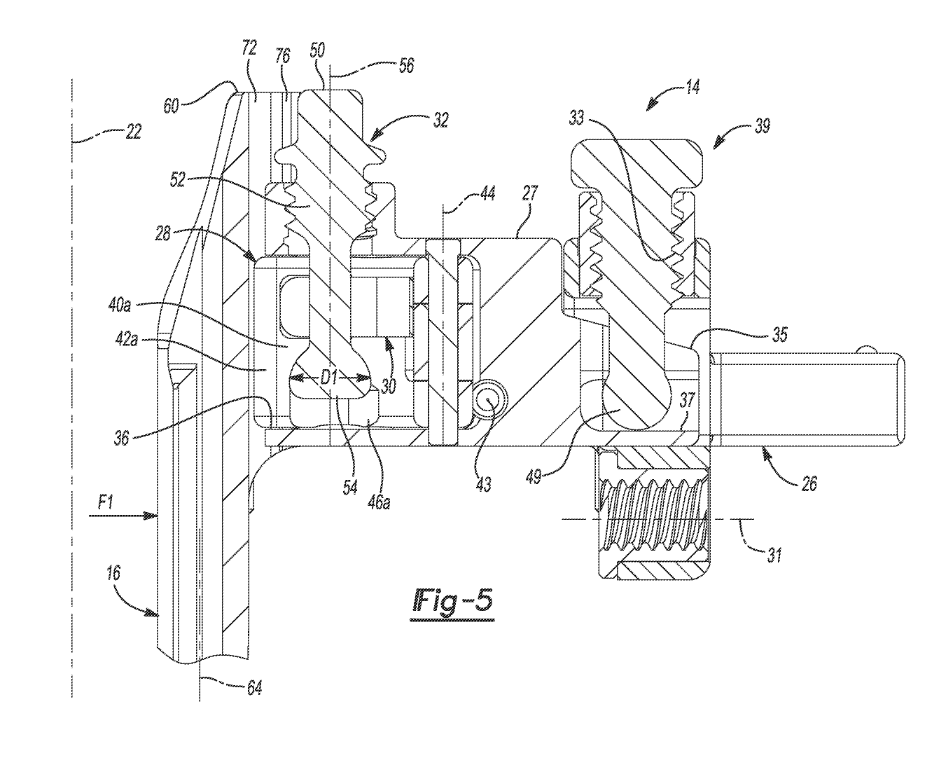

The connector housing 27 may be pivotably mounted to the carrier 26 for rotation about a third axis of rotation 43. The third axis of rotation 43 may be substantially perpendicular to the first longitudinal axis 22 and substantially perpendicular to the second axis of rotation 31. In this regard, as illustrated in FIG. 5, the carrier 26 may include a threaded aperture 33, and the connector housing 27 may include a stem portion 35 having an open-ended channel 37. The channel 37 may define a cross-sectional area A1. A screw, bolt, or other similar mechanical fastener 39 may be threadably received by the threaded aperture 33 and extend into the channel 37. The fastener 39 may include an end portion 49 having a cross-sectional area A2 sized and shaped so as rotatably mate within the channel 37. Rotating the fastener 39 may cause the end portion 49 to contact the stem portion 35 of the connector housing 27, and thereby cause the connector housing to rotate or pivot about the third axis of rotation 43.

The connector 28 may be coupled to the connector housing 27. In this regard, the connector housing 27 may further include an opening 36. In an assembled configuration, the opening 36 may generally face the central opening 18 of the frame 12. The connector 28 may be located within and extend from the opening 36, and may include a first arm 40a and a second arm 40b. The first and second arms 40a, 40b may be operable to pivot about a fourth axis of rotation 44 that is substantially parallel to the first longitudinal axis 22 of the frame 12. The first and second arms 40a, 40b may each include an outwardly projecting flange or lipped portion 42a, 42b, respectively, and concave or otherwise recessed portions 46a, 46b, respectively. As will be explained in more detail below, the outwardly projecting flange or lipped portions 42a, 42b may secure the blade 16 to the connector 28.

The biasing member 30 may be located between the first and second arms 40a, 40b and may be operable to bias the connector 28 into engagement with the blade 16. As illustrated, in one configuration the biasing member 30 may be a substantially U-shaped leaf spring disposed between the first and second arms 40a, 40b and operable to bias the first arm 40a relative to the second arm 40b. In this regard, the biasing member 30 may be operable to bias the first arm 40a away from the second arm 40b and about the fourth axis of rotation 44. While the biasing member 30 is shown as being a leaf spring, it will be appreciated that the biasing member 30 may have other configurations, such as a helical spring, within the scope of the present disclosure. It will also be appreciated that the connector 28 may be formed from a material having spring-like characteristics, such that construct of the connector 28 biases the first arm 40a away from the second arm 40b in a leaf spring-like manner.

The locking element 32 may include a head portion 50, an intermediate portion 52, and a lock portion 54. The locking element 32 may be rotatably coupled to the carrier 26 for rotation about a fifth axis of rotation 56. The fifth axis of rotation 56 may be substantially parallel to the fourth axis of rotation 44. As illustrated, in one configuration the head portion 50 is located at the upper side 23 of the frame 12 and includes a driving feature such as a hex head, a socket, or other multi-lobular aperture for selectively rotating the locking element 32 about the fifth axis of rotation 56. The intermediate portion 52 extends between the head portion 50 and the lock portion 54 and may be threadably engaged with the connector housing 27.

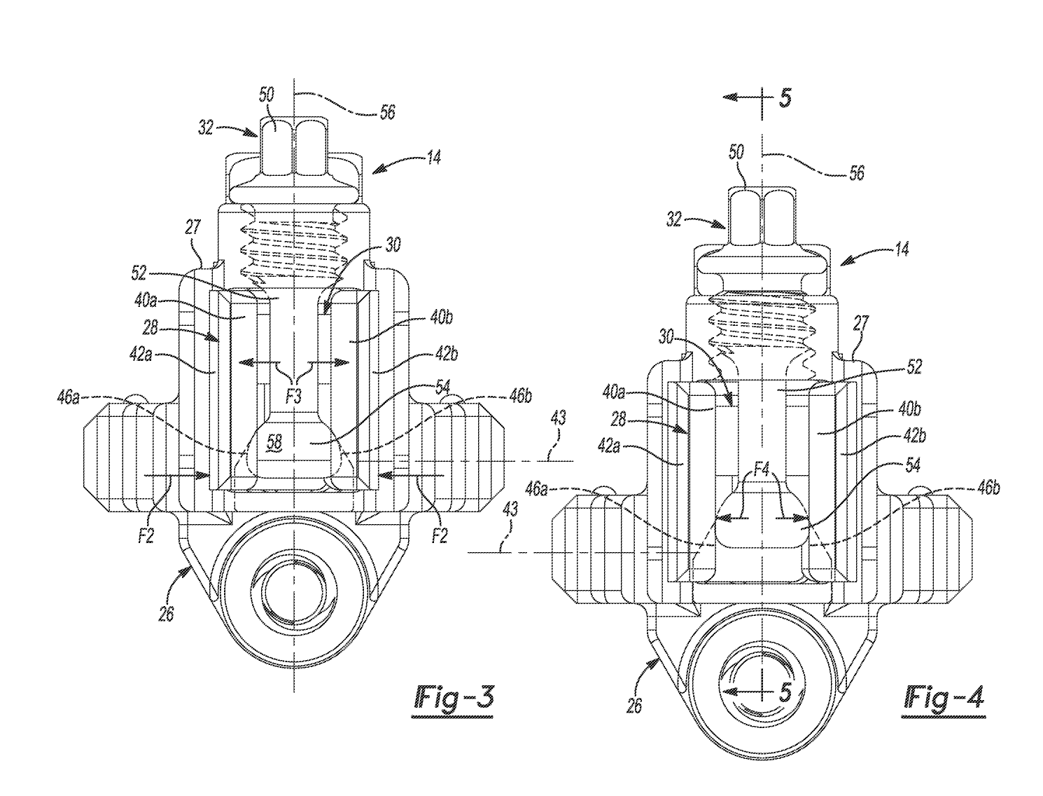

As illustrated in FIGS. 3 and 4, the lock portion 54 may include a substantially spherical or dome-like surface 58 located between the first and second arms 40a, 40b of the connector 28. The dome-like surface 58 may have a diameter Dl. With reference to FIG. 3, in a first or unlocked configuration, the surface 58 of the lock portion 54 may be located in, and adjacent to, the recessed portions 46a, 46b of the first and second arms 40a, 40b. As will be explained in more detail below, in the unlocked configuration, the locking element 32 may allow translation of the blade 16 relative to the first and second arms 40a, 40b in a direction substantially parallel to the fifth axis of rotation 56. In a locked configuration, the locking element 32 may prevent translation of the blade 16 relative to the first and second arms 40a, 40b in a direction substantially parallel to the fifth axis of rotation 56. As will be explained in more detail below, by rotating the locking element 32 about the fifth axis of rotation 56, the threaded engagement between the locking element 32 and the connector housing 27 may cause the locking element 32 to move along (i.e., parallel to) the fifth axis of rotation 56, such that the lock portion 54 exits the recessed portions 46a, 46b. As illustrated in FIG. 4, when the lock portion 54 exits the recessed portions 46a, 46b, the dome-like surface 58 may force the first arm 40a away from the second arm 40b and into the locked configuration.

The blade 16 may extend longitudinally between a proximal end 60 and a distal end 62, along a second longitudinal axis 64. As illustrated in FIG. 1, in the assembled configuration, the second longitudinal axis 64 of the blade 16 may be substantially parallel to the fifth axis of rotation 56, such that a first side 66 of the blade 16 faces the opening 18, and a second side 68 of the blade 16 faces the connector 28. The first side 66 of the blade 16 may be substantially concave, such that the first sides 66 of the blades 16 collectively define a circular shape, as illustrated in FIG. 2.

The second side 68 of the blade 16 may include a mounting portion 70. In one configuration, the mounting portion 70 is located at the proximal end 60 of the blade 16. The mounting portion 70 may include a first arm 72 and a second arm 74. The first arm 72 may include a first lipped or flanged portion 76 and the second arm 74 may include a second lipped or flanged portion 78, such that the first and second arms 72, 74 and the first and second flanged portions 76, 78 collectively define a substantially U or C-shaped mounting portion. Specifically, the mounting portion 70 may define a substantially C-shaped cross section extending along the second longitudinal axis 64 of the blade 16. The C-shaped cross section, including the first and second arms 72, 74 and the first and second flanged portions 76, 78, may have a length extending less than or equal to fifty millimeters in the direction substantially parallel to the second longitudinal axis 64. In one particular configuration, the C-shaped cross section may have a length equal to ten millimeters. As explained in more detail below, the length of the C-shaped cross section may in part define a distance by which the blade 16 can translate or otherwise move relative to the connector 28 along the second longitudinal axis 64.

In the assembled configuration, the mounting portion 70 of the blade 16 may be adjustably coupled to the connector 28. In this regard, the first and second arms 72, 74 of the mounting portion 70 may be coupled to the first and second arms 40a, 40b, respectively, of the connector 28, such that the first and second flanged portions 76, 78 mate with the lipped portions 42a, 42b, respectively, of the connector 28.

Operation of the surgical retractor 10 will now be described in more detail. The surgical retractor 10 may be positioned over an incision or other surgical access site such that the first longitudinal axis 22 of the central opening 18 is substantially aligned with the incision. At least one of the blades 16 may be positioned within the opening 18 and/or the incision such that the second longitudinal axis 64 of the blade 16 is substantially aligned, or otherwise parallel to, the first longitudinal axis 22 of the central opening. The blade 16 may be moved in a direction substantially perpendicular to the second longitudinal axis 64 and substantially perpendicular to the third axis of rotation 43, such that the mounting portion 70 engages the connector 28. In this regard, as illustrated in FIG. 5, the user may apply a force F1 on the blade 16 in a direction extending away from, and substantially perpendicular to, the first longitudinal axis 22, such that the first and second flanged portions 76, 78 of the mounting portion 70 contact the first and second lipped portions 42a, 42b of the connector 28. As illustrated in FIG. 3, the first and second flanged portions 76, 78 may apply a force F2 on the arms 40a, 40b of the connector 28 that is opposite a biasing force F3 of the biasing member 30. The force F2 may overcome the biasing force F3, such that the first arm 40a moves toward the second arm 40b and/or about the fourth axis of rotation 44. The mounting portion 70 may be moved away from, and substantially perpendicular to, the first longitudinal axis 22 until the mounting portion 70 is coupled to the connector 28.

With the mounting portion 70 coupled to the connector 28, the distance between the distal end 62 of the blade 16 and the connector 28 can be adjusted along the second longitudinal axis 64 by sliding or otherwise translating the mounting portion 70 relative to the connector 28 along the C-shaped cross section of the mounting portion 70. In this regard, the C-shaped cross section of the mounting portion 70 may include a closed end that contacts a distal end of the arms 40a, 40b to limit the amount of relative movement between the blade 16 and the connector 28 along the second longitudinal axis 64. When the distal end 62 of the blade 16 has been positioned at a desired location relative to the incision, the user may adjust the locking element 32 to secure the mounting portion 70 relative to the connector 28 and prevent movement of the blade 16 along the second longitudinal axis 64. Specifically, the user may rotate the locking element 32 about the fifth axis of rotation 56 from the unlocked position to the locked position, as described above, such that the lock portion 54 exits the recessed portions 46a, 46b of the first and second arms 40a, 40b, respectively. As illustrated in FIG. 4, the lock portion 54, including the surface 58, may apply a force F4 on the first and second arms 40a, 40b that generates and/or increases a normal force between the arms 40a, 40b and the arms 72, 74. The normal force between the arms 40a, 40b and the arms 72, 74 may increase a frictional force between the arms 40a, 40b and the arms 72, 74 that is operable to prevent the blade 16 from moving in a direction substantially parallel to the second longitudinal axis 64 of the blade 16.

When it is desired to adjust the position of the mounting portion 70 relative to the connector 28 along the second longitudinal axis 64, or otherwise remove the blade 16 from the connector 28, the user may rotate the locking element 32 from the locked position to the unlocked position such that the lock portion 54 is received between the recessed portions 46a, 46b of the first and second arms 40a, 40b. In this way, the frictional force between the arms 40a, 40b of the connector 28 and the arms 72, 74 of the mounting portion 70 is reduced, allowing the user to slide the mounting portion 70 relative to the connector 28.

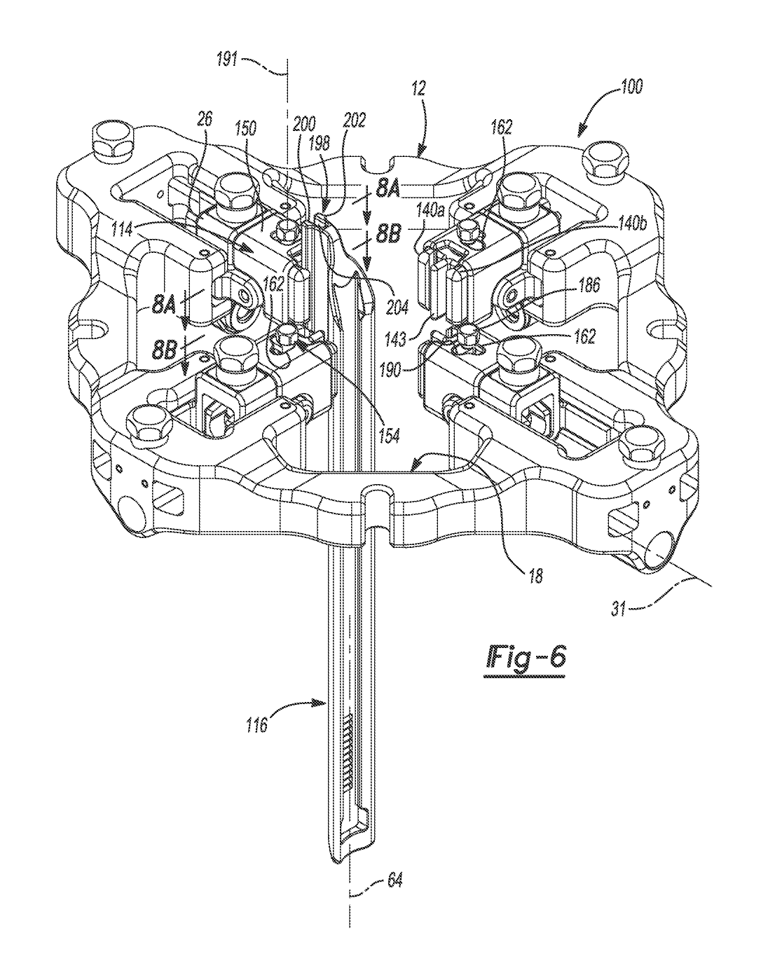

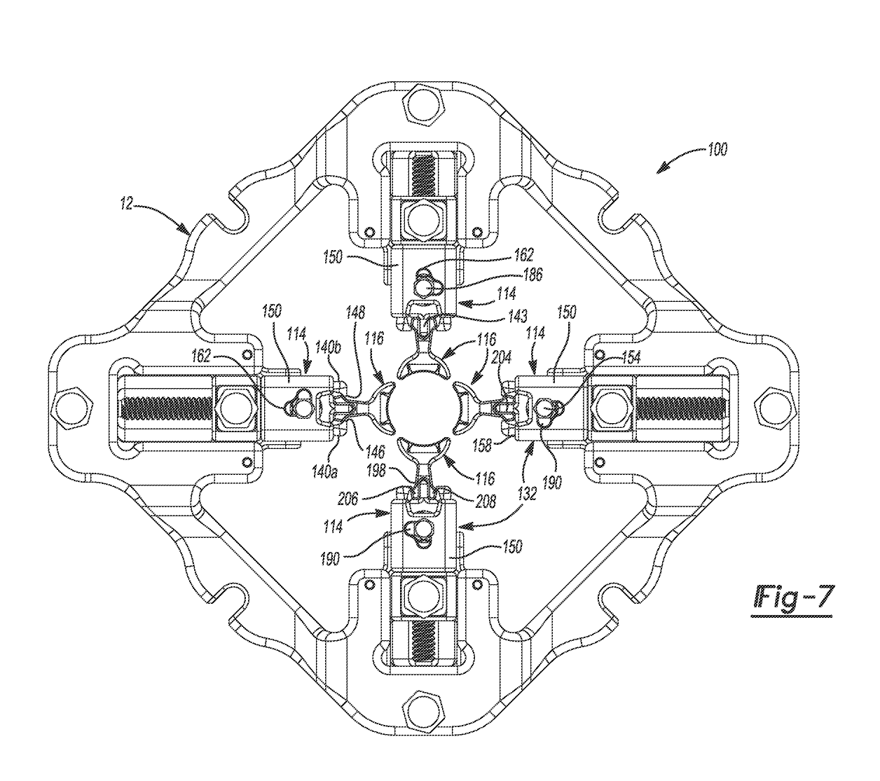

With reference to FIGS. 6 through 9B of the drawings, another configuration of a surgical retractor constructed in accordance with the principles of the present disclosure is illustrated and identified at reference character 100. The surgical retractor 100 may be substantially similar to the surgical retractor 10, except as otherwise provided herein. Accordingly, similar features will not be described again in detail.

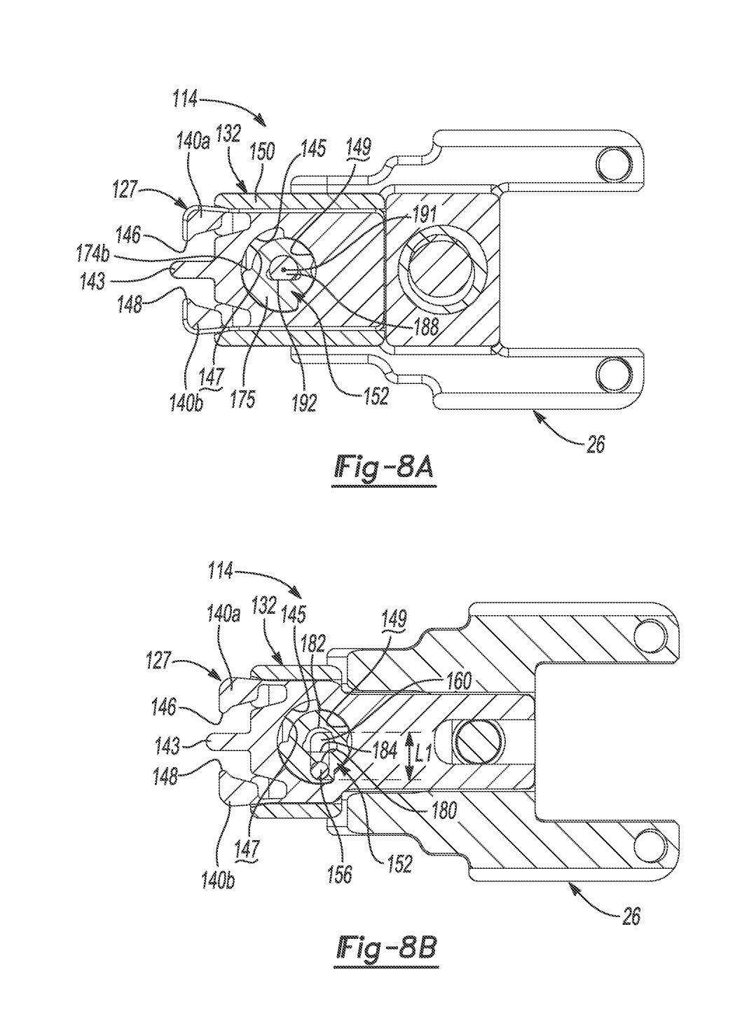

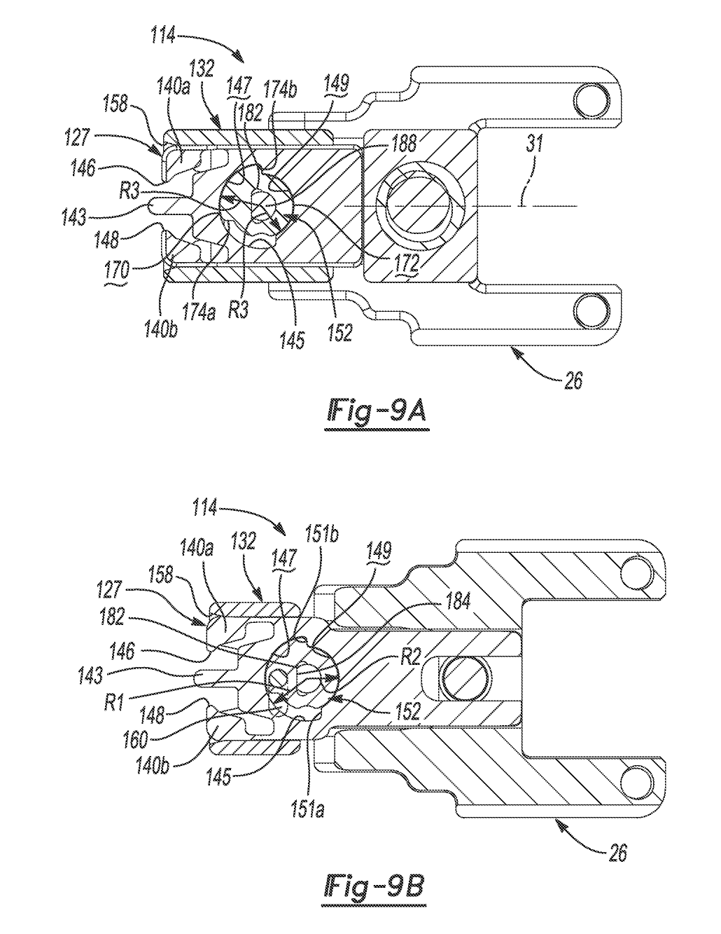

The surgical retractor 100 may include a coupling arrangement 114 and a blade 116. The coupling arrangement 14 may include a connector 127 and a locking assembly 132. As illustrated in FIGS. 8A through 9B, the connector 127 may be adjustably mounted to the carrier 26, and may include a first arm 140a, a second arm 140b, a guide 143, and an aperture 145. The first and second arms 140a, 140b may be pivotably mounted to and/or integrally formed with the connector 127, and may include an inwardly projecting flange or lipped portion 146, 148, respectively, for securing the blade 116 to the connector 127. The guide 143 may be a flange or fin-type element extending between and substantially parallel to the first and second arms 140a, 140b. The guide 143 may be integrally formed with the connector 127. The aperture 145 may include a first and second arcuate surfaces 147, 149 and first and second stop surfaces 151a, 151b (FIG. 9B). The first and second arcuate surfaces 147, 149 may be concave and include first and second radii R1, R2, respectively. The first and second stop surfaces 151a, 151b may extend from and between the first and second arcuate surfaces 147, 149. Operation of the connector 127 will be described in more detail below.

The locking assembly 132 may include a housing 150, a stem element 152, a locking element 154, and a pin member 156. The housing 150 may include an opening 158, a first aperture 160 and a second aperture 162. The opening 158 may be sized and shaped to adjustably receive the connector 127. The first aperture 160 may be a substantially oblong slot located on a first side of the housing 150. The first aperture 160 may include a length L1 extending in a direction substantially perpendicular to the first longitudinal axis 22 and substantially perpendicular to the second axis of rotation 31. The second aperture 162 may be a generally L-shaped aperture located on a second side of the housing 150, generally opposite the first side of the housing. In this regard, the first side of the housing 150 may be substantially parallel to the second side of the housing 150.

The stem element 152 may include a cross-sectional shape that is similar to the shape of the aperture 145 such that, in an assembled configuration, the stem element 152 is received within the aperture 145. In this regard the stem element 152 may include third and fourth arcuate surfaces 170, 172 (FIG. 9B) and third and fourth stop surfaces 174a, 174b. The third and fourth arcuate surfaces 170, 172 may be convex and include third and fourth radii R3, R4, respectively. The third arcuate surface 170 may substantially define a cam portion 175 (FIG. 8A) of the stem element 152. The first and second stop surfaces 151a, 151b may extend from and between the third and fourth arcuate surfaces 170, 172. In an assembled configuration, the stem element 152 may be rotatably received in the aperture 145 of the connector 127.

A first end of the stem element 152 may include a first bore 180 operable to receive the pin member 156. In one configuration, the first bore 180 may be disposed in the cam portion 175 of the stem element 152. A second end of the stem element 152 (generally opposite the first end of the stem element 152) may include a second bore 182 operable to receive the locking element 154. The second bore 182 may include a flat 184. The locking element 154 may include a head portion 186, a stem portion 188, and an arm portion 190. The head portion 186 may include a hex head, a socket, or other driving feature for operatively rotating the locking element 154 with a wrench or other suitably driving tool. The stem portion 188 may extend from the head portion 186 and include a flat 192. The arm portion 190 may extend generally perpendicularly from the stem portion 188.

In an assembled configuration, the pin member 156 may be mounted within the first bore 180 of the stem element 152. The locking element 154 may be mounted within the second bore 182 of the stem element 152, such that the flat 184 is aligned with the flat 192 to prevent the locking element 154 from rotating within the second bore 182. In the assembled configuration, the arm portion 190 of the locking element 154 may be located adjacent to the cam portion 175 of the stem element 152. The stem element 152 may be mounted within the aperture 145 of the connector 127 for rotation about a seventh axis of rotation 191. The connector 127 may be movably or otherwise adjustably mounted to the housing 150, such that the first and second arms 140a, 140b extend from the opening 158 of the housing 150. In the assembled configuration, the pin member 156 may extend into the first aperture 160 of the housing 150, and the arm portion 190 of the locking element 154 may extend into the second aperture 162 of the housing 150.

The blade 116 may be substantially similar to the blade 16. Accordingly, like reference numerals will be used to describe similar features. The blade 116 may include a mounting portion 198 having a generally Y-shaped cross section. In this regard, the mounting portion 198 may include a first arm 200, a second arm 202, and an opening or slot 204 extending between the first and second arms 200, 202. The first arm 200 may include a first outwardly extending lip or flange portion 206 and the second arm 202 may include a second outwardly extending lip or flange 208.

Operation of the surgical retractor 100 will now be described in more detail. Operation of the surgical retractor 100 may be substantially similar to the operation of surgical retractor 10. Accordingly, only the differences will be described in detail herein.

The blade 116 may be moved in a direction substantially perpendicular to the second longitudinal axis 64, such that the mounting portion 198 engages the connector 127. In this regard, the user may apply a force F1 on the blade 116 in a direction extending away from, and substantially perpendicular to, the first longitudinal axis 22 until the first and second arms 200, 202 of the mounting portion 198 contact the first and second arms 140a, 140b of the connector 127, and the guide 143 is received by the slot 204.

With the mounting portion 198 coupled to the connector 127, the distance between the distal end 62 of the blade 116 and the connector 127 can be adjusted along the second longitudinal axis 64 by sliding or otherwise translating the mounting portion 198 relative to the connector 127. In this regard, the slot 204 of the mounting portion 198 may include a closed end. The closed end may contact the guide 143 to limit the amount of relative movement between the blade 116 and the connector 127. When the distal end 62 of the blade 16 has been positioned at a desired location, the user may rotate the stem element 152 about the seventh axis of rotation 191, and within the aperture 145 of the connector 127. Specifically, the user may use a wrench, or other suitable driving tool, to rotate the locking element 154, and in turn the stem element 152. As the stem element 152 is rotated, the pin member 156 may slide or otherwise translate within the first aperture 160, and the arm portion 190 of the locking element 154 may slide or otherwise translate within the second aperture 162 of the housing 150. The pin member 156 and the locking element 154 may, in turn, cause the housing 150 to slide or otherwise translate relative to the connector 127. The housing 150 may force the first arm 140a towards the second arm 140b, and thereby secure the mounting portion 198 of the blade 116 between the first and second arms 140a, 140b, to prevent the blade 116 from moving in a direction substantially parallel to the second longitudinal axis 64.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

1. A surgical retractor comprising:
a retractor frame;
a plurality of blades for engaging tissue, the plurality of blades defining a longitudinal blade axis through the retractor frame; and
a coupling arrangement carried by the retractor frame and releasably coupling a first blade of the plurality of blades with the retractor frame through translation of the first blade in a direction generally perpendicular to the longitudinal axis, the coupling arrangement including a first driving member configured to activate translation of the coupling arrangement in a direction generally perpendicular to the longitudinal blade axis.

2. The surgical retractor of claim 1, wherein the coupling arrangement is operative to selectively allow translation of the first blade relative to the coupling arrangement along the longitudinal axis of the first blade.

3. The surgical retractor of claim 1, the first blade includes a connector portion and wherein the coupling arrangement includes a first arm and a second arm biased into engagement with the connector portion.

4. The surgical retractor of claim 3, wherein the biasing member is integrally formed with the first and second arms.

5. The surgical retractor of claim 3, wherein the connector portion slidably engages the first and second arms for translation along the longitudinal axis.

6. The surgical retractor of claim 5, wherein the coupling arrangement includes a locking member for selectively preventing translation of the connector portion relative to the first and second arms.

7. The surgical retractor of claim 6, wherein the locking member is rotatably mounted to the connector element for rotation between a locked position and an unlocked position.

8. The surgical retractor of claim 7, wherein the first and second arms are biasingly engaged with the locking member in the locked position and are spaced apart from the locking member in the unlocked configuration.

9. The surgical retractor of claim 7, wherein the locking member is rotatable about an axis of rotation substantially parallel to the longitudinal axis.

10. The surgical retractor of claim 1, wherein the coupling arrangement is movably coupled to the retractor frame for translation along a second axis substantially perpendicular to the longitudinal axis, wherein the first driving member engages a second driving member to enable translation of the coupling arrangement along the second axis relative to the retractor frame.

11. The surgical retractor of claim 2, wherein the coupling arrangement cooperates with the first blade to limit translation of the first blade in a direction parallel to the longitudinal axis.

12. A surgical retractor comprising:
a retractor frame;
a plurality of blades for engaging tissue, each blade including a mounting portion having a pair of blade arms and the plurality of blades defining a longitudinal axis through the retractor frame; and
a coupling arrangement carried by the retractor frame and releasably coupling a first blade of the plurality of blades with the retractor frame through translation of the first blade in a direction generally perpendicular to the longitudinal axis, the coupling arrangement including a pair of coupling arms moveable relative to one another in a direction generally perpendicular to the longitudinal axis of the first blade, the coupling arrangement further including a first driving member rotatably engaging portions of the coupling arrangement to activate movement of the coupling arrangement.

13. The surgical retractor of claim 12, wherein the coupling arrangement further includes a biasing member for biasing the coupling arms relative to one another.

14. The surgical retractor of claim 13, wherein the biasing member biases the coupling arms away from one another.

15. The surgical retractor of claim 12, wherein the coupling arms are moveable relative to one another between a first orientation for receiving the first blade in the direction generally perpendicular to the longitudinal axis, and a second orientation for retaining the first blade.

16. The surgical retractor of claim 15, wherein the coupling arrangement further includes a locking mechanism for locking the coupling arms in the second orientation.

17. The surgical retractor of claim 13, wherein the biasing member is a leaf spring.

18. A surgical retractor comprising:
a retractor frame;
a plurality of blades for engaging tissue, each blade defining a longitudinal axis and including a mounting portion having a pair of blade arms; and
a corresponding plurality of coupling arrangements carried by the retractor frame, each coupling arrangement operative to releasably couple a corresponding blade of the plurality of blades with the retractor frame through translation of the corresponding blade in a direction generally perpendicular to the longitudinal axis of the corresponding blade, wherein the translation of each coupling arrangement of the plurality of coupling arrangements is activated through rotation of a corresponding plurality of first driving members, each first driving member associated with a coupling arrangement of the plurality of coupling arrangements.

19. The surgical retractor of claim 18, wherein each coupling arrangement includes a first coupling arm and a second coupling arm, and wherein the first coupling arm is moveable relative to the second coupling arm in a direction generally perpendicular to the longitudinal axis of each blade.

20. The surgical retractor of claim 19, wherein the first and second coupling arms are biased into engagement with the mounting portion.
